(19) United States
(12) Patent Application Publication
(10) Pub. No.: US 2007/0153971 A1
(43) Pub. Date: Jul. 5, 2007

(54) Controlled cardiac computed tomography

(76) Inventors: Chenglin Wang, Iowa City, IA (US); Ying Liu, Blacksburg, VA (US)

Correspondence Address: Chenglin Wang, 4029 El Paso Dr., Iowa City, IA, 52246, US

(21) Appl. No.: 11/646,669

(22) Filed: Dec. 28, 2006

Related U.S. Application Data
(60) Provisional application No. 60/754,677, filed on Dec. 30, 2005.

(51) Int. Cl. A61B 6/00; G01N 23/00; G21K 1/12; H05G 1/60

(52) U.S. Cl. 378/8

(57) ABSTRACT

Cardiac computed tomography (CT) has been a hot topic for years because of the clinical importance of cardiac diseases and the rapid evolution of CT systems. In this application, we disclose a novel strategy for controlled cardiac CT (CCCT) that may effectively reduce image artifacts due to cardiac and respiratory motions and reduce the scan time. Our approach is radically different from existing ones and is based on controlling the x-ray source rotation velocity and powering status in reference to the cardiac motion. By such a control-based intervention the data acquisition process can be optimized for cardiac CT in the cases of periodic and quasi-periodic cardiac motions. Specifically, we present the corresponding coordination/control schemes for either exact or approximate matches between the ideal and actual source positions.

### CROSS-REFERENCE TO RELATED APPLICATION

This application claims priority to U.S. Provisional Application No. 60/754,677 filed on Dec. 30, 2005 and hereby incorporated by reference in its entirety.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DIS APPENDIX

Not applicable.

### BACKGROUND OF INVENTION

Cardio-vascular disease is the number one killer in the Western world. It is responsible for 1 of every 2.6 deaths in the United States. Cardio-vascular disease was listed as a primary or contributing cause on about 1,408,000 death certificates annually. One in five persons has some form of cardiovascular diseases in the United States, account for 64,400,000 Americans (http://www.americanheart.org/). In 2004, the estimated cost of cardio-vascular diseases is $368.4 billion.

Electron-beam CT (EBCT) is a unique mode of medical x-ray CT, which is for early screening of coronary artery diseases [1]. There are three major limitations with the current EBCT techniques. First, it is not in cone-beam geometry, and hence it can only acquire a limited number of transverse slices through the heart. It has become clear that the multi-slice/cone-beam scanning is much more advantageous for a good portion or even the whole volume of the heart to be contained in a cone-beam, and reconstructed from a rapidly acquired dataset for both high temporal resolution and excellent anatomical consistency. Second, the x-ray spot in EBCT is not sufficiently intensive to produce the image quality that the mechanical rotation based scanners can achieve, which compromises contrast resolution in reconstructed images. Third, the EBCT scanner is expensive and monstrous. As such, EBCT is far less accessible and less cost-effective than the main stream CT scanner that utilizes a mechanically rotated x-ray source.

Given the aforementioned limitations of the EBCT, multi-slice/cone-beam CT has been growing into a strong competitor of the EBCT [2,3]. There are two major problems with current gating based cardiac CT methods. First, these existing methods are passive in their nature, and require that the cardiac motion and the x-ray source rotation must be at favorable relative frequencies; otherwise, the data sectors to be assembled for a complete dataset would span a wide range of the cardiac status leading to a compromised image quality, or the data acquisition time would be too long to be practical (in a most unfavorable case, it could be impossible to acquire a complete dataset). Second, even in the favorable cases, retrospectively reconstructed cardiac images still suffer from substantial motion blurring because in practice each projection sector covers a projection angular range of a substantial length. Within such an angular range, the heart moves appreciably, especially when it is not in a relative stationary phase. As a benchmark, we routinely achieve <0.3 mm spatial resolution in spiral CT of the temporal bone where the motion magnitude is much less than that of the heart. On the other hand, the spatial resolution with cardiac CT is at best in the millimeter domain. It is claimed that the spatial resolution with our invented controlled cardiac CT can be significantly better than that with current cardiac CT, and eventually made to be comparable to that with temporal bone CT.

To understand etiology and pathogenesis of the human cardio-vascular diseases as well as develop prevention and treatment strategies, small animals have become common laboratory models. To use these animal models fully, the extension of cardiovascular imaging from patients to small animals, such as mice and rats, is imperative. Because of the small size and high heart rate of a mouse, high spatial and temporal resolutions are required. Currently, an x-ray micro-CT scanner takes at least 20 s to acquire a full dataset. Hence, cardiac micro-CT represents a daunting challenge in this field, requiring major interdisciplinary efforts. The recent small animal micro-CT work performed at Duke University [4] showed the feasibility of respiratory and cardiac gated micro-CT but their system runs very slowly (“not well suited for high throughput”), and requires that the mouse is placed vertically and rotated asynchronously, violating the natural physiological conditions.

### BRIEF SUMMARY OF THE INVENTION

Over the past several decades, tremendous progress has been made in the fields of computed tomography (CT) and automatic control, largely driven by the need for real-time performance in these fields. However, the synergy between CT and control has not been effectively utilized in the medical imaging arenas until the recent project on bolus-chasing CT angiography that was initiated at the University of Iowa and funded by NIH/NIBIB [5-8]. While bolus-chasing CTA project couples adaptive control and real-time CT to translate the patient table for synchronization of a moving bolus and the imaging aperture of a scanner, this invention is to control the X-ray source rotation based on the ECG signal or similar information to achieve unprecedented cardiac CT capabilities [9-11]. The development of such controlled cardiac CT (CCCT) systems has just become technically feasible and commercially profitable for not only medical imaging but also micro-CT of small animals. We emphasize that the current gating based cardiac CT methods do not have the leverage to manipulate or select the source rotation speed adaptively, hence they miss a major opportunity for optimization of the imaging performance. Our invention is to make CCCT a vital mode in cardiac studies. Here we disclose our technologies that can be used by those who are experts in the fields to (1) develop schemes for adaptive control or real-time selection of the x-ray source rotation to minimize the effects of cardiac motions and the data acquisition time under realistic hardware constraints, assuming periodic and quasi-periodic cardiac motion respectively; (2) optimize CT algorithms for cardiac reconstruction in both fan-beam and cone-beam geometry; (3) integrate and evaluate the control and imaging techniques into real systems for cardiac imaging.

### BRIEF DESCRIPTION OF DRAWINGS

The accompanying drawings, which are incorporated in and constitute a part of this specification, illustrate embodiments of the invention and together with the description, serve to explain the principles of the invention:

FIG. 1. Illustration for one embodiment of the invented controlled cardiac CT/micro-CT system.

FIG. 2. Illustration for one embodiment of the source rotation control strategy.

FIG. 3. Cardiac CT implemented using a constant source rotation velocity and the same set of projection angles for all the volumetric levels of interest.

FIG. 4. Cardiac CT implemented using a constant source rotation velocity and different sets of projection angles for various volumetric levels of interest.

FIG. 5. Relationships among the total scanning time, maximum velocity and maximum acceleration for controlled cardiac CT. (a) Plots showing the total time needed to cover three angles exactly versus the corresponding maximum velocity/maximum acceleration in one case study, and (b) plots showing velocity and acceleration changes versus the total time. The greater the allowed acceleration is, the shorter the total time will be, vice versa.

FIG. 6. Simulation results of controlled cardiac CT with multi-level multi-angle quasi-periodic tracking. The projection angles are “randomly” ordered for each of the pre-specified levels of the cardiac function. The velocity of the x-ray source rotation must be variable for an accurate solution.

FIG. 7. Simplified heart volume function used in our simulation. There are 5 phases for cardiac motion. Period between 0.3-0.4 s is the least motion phase, which was used for cardiac imaging.

FIG. 8. Reconstructed images in different cardiac phases (heart beat period=0.6 s) using the current multi-segment algorithm. (a) A reconstructed image with ideal data segments for t=0.3-04 s, and (b) that with gaps between data segments for t=0.3-0.38 s. (a) is the best (ideal data segments), while (b) is compromised.

FIG. 9. Reconstructed images with a fixed source velocity for GE Light scanner and an optimally selected source velocity in the case of heart rate 78 bpm. (a)-(f) Images from source rotation periods 0.5, 0.6, 0.7, 0.8, 0.9, and 1.0 s per source rotation which are permitted by a GE Light speed CT scanner, and (g) the corresponding image with our selected source period 0.43 s. Clearly, (g) is significantly better than (a)-(f).

FIG. 10. Reconstructed images with different velocities as determined by our selectable velocity method, assuming that the heart beat period is 0.6 s. (a) An image with a slower source rotation velocity Pc=0.5 s (m=1), and (b) that with a faster source rotation period Pc=0.33 s (m=2). Clearly, (b) is superior to (a) because of faster rotation.

FIG. 11. Reconstructed images with different scan times as determined by our selectable velocity method. (a) An image with a scan time Pc=5P, and (b) that with a scan time Pc=36P. Clearly, (b) is better than (a) because of longer scan time.

### DETAILED DESCRIPTION OF THE INVENTION

One or more exemplary embodiments are now described in detail herein below. Referring to the drawings, like numbers indicate like parts throughout the views. As used in the description herein, the meaning of “a,” “an,” and “the” includes plural reference unless the context clearly dictates otherwise. Also, as used in the description herein, the meaning of “in” includes “in” and “on” unless the context clearly dictates otherwise. Finally, as used in the description herein, the meanings of “and” and “or” include both the conjunctive and disjunctive and may be used interchangeably unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

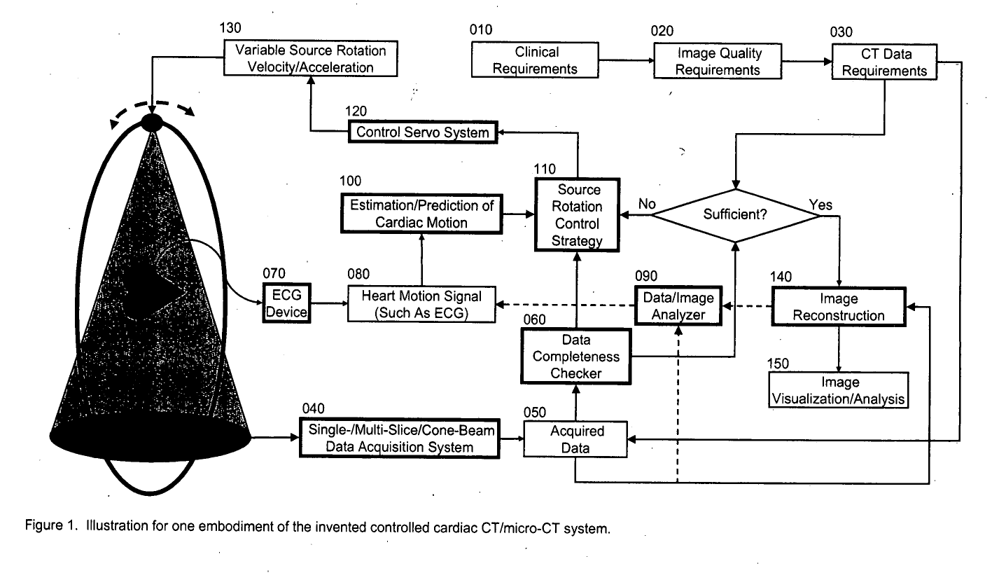

FIG. 1 illustrates one embodiment of the invented controlled cardiac CT/micro-CT system. Based on the clinical requirements (010), the image quality requirements (020) are defmed, which are translated to the CT data requirements (030). Then, a single-/multi-slice/cone-beam data acquisition system (040) starts collecting data. The acquired data (050) is constantly processed by a data completeness checker (060) to reveal the data missing status in reference to the data requirements (030). The cardiac motion is monitored by an ECG device (070) which produces the cardiac motion signal (080). Such a signal may be also extracted from a data/image analyzer (090). Based on the cardiac motion signal (080), the cardiac motion pattern will be estimated/predicted (100). If a sufficient amount of data has not been accumulated yet, according to our source rotation control strategy (110) the x-ray source will be optimally steered as needed by a control servo system (120) at variable source rotation velocity/acceleration (130) to fill in the missing data gaps. Finally, image reconstruction (140) is completed for further analysis and visualization (150) The computing resources are important for implementation of these functionalities but they are not shown here for clarity.

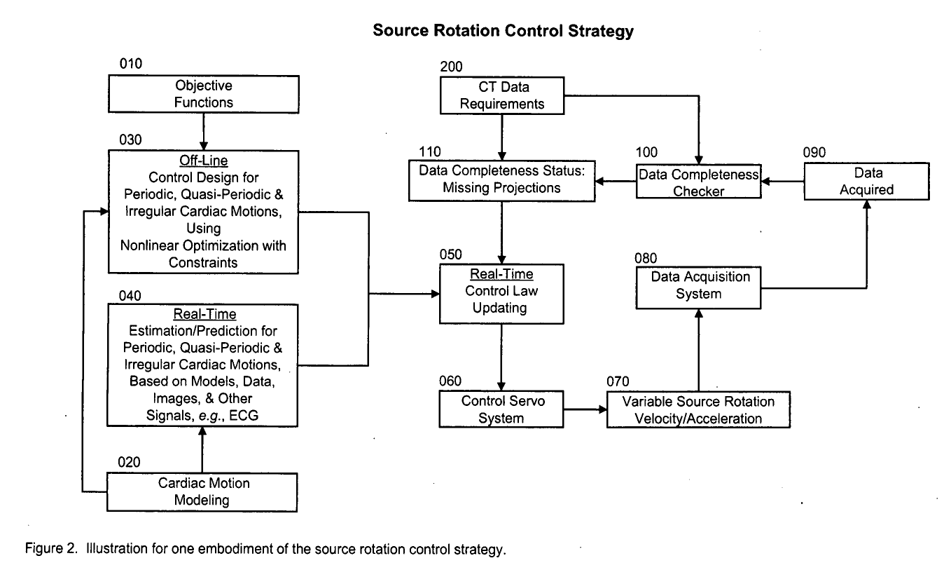

FIG. 2 illustrates one embodiment of the source rotation control strategy. The objective functions (010) are set in reference to a cardiac motion model (020). The off-line design (030) is first performed to optimize the control strategies in various settings based on the cardiac motion modeling. The real-time estimation/prediction (040) is done to capture the trend of the cardiac motion, which is also based on the cardiac motion modeling. The off-line optimized guidelines and the real-time analysis results are combined to update our control law (050) in real-time. Under the control law, the control servo system (060) steers the source rotation at a variable velocity/acceleration (070) for the data acquisition system (080) to collect projection data up to the CT data requirements. At any instant, acquired data (090) are analyzed by the data completeness checker (100) to update the data completeness status (110) with respect to the CT data requirements (200). The computing resources are not shown here for brevity.

It is underlined that the references of ours cited therein provide more details on our invention, and should be considered as an integrated part of this patent application. Also, the description in this part should be interpreted as exemplary instead of restrictive. We recognize that variants of this technology are clearly possible in the spirit of this application including the cited references of ours.

A. Adaptive Control

It is recognized that the cardiac motion is certainly complicated by other factors, especially the respiratory motion. However, in the following we will focus on the cardiac motion for two reasons. Primarily, the methodology applies similarly to the combination of cardiac and respiratory motions. Secondarily, the cardiac motion is the dominating component to be corrected for cardiac CT in most cases.

For a heart in a periodic cardiac motion, it is relatively easy to verify its regularity and estimate its period. For example, it can be done based on analysis of a number of cycles of the ECG signal of a patient. We will start with the case of periodic motion based control as a basis for quasi-periodic motion based control, which is our desired model for cardiac CT.

A.1. Period Cardiac Motion

To convey the idea without unnecessary complication, we will first focus on one volume level in the expanding or contracting phase of the cardiac motion. Let v(t)=v(t+P) be the heart volume which is periodic with period P, and v(t₁) be the heart level that we are interested in and

$${a}_{l} = \frac{l - 1}{p} 2 π , l = 1 , … , p ,$$

be p angles at which the x-ray source has to be on. It is recognized that for the same cardiac volume level the structure of the heart may be slightly different in the expanding and contracting phases. Without loss of generality, we assume such a difference is substantial and requires that the heart of a pre-specified volume must be sampled at the consistent phases of the ECG signal; for example, in the expanding phase.

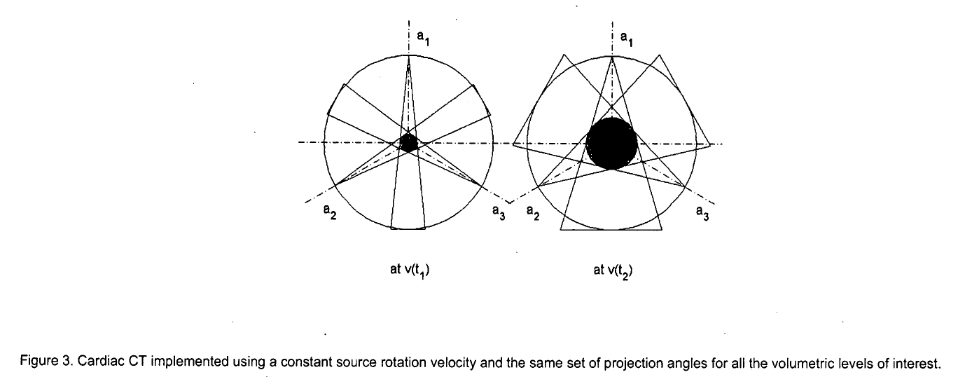

Ideally, the problem might be solved with a pre-specified constant source velocity. Recall that v(t)=v(t+P) is the heart volume, αₗ, l=1, 2, . . . , p, p evenly spaced angles and s(t) the source angle. The problem we have to solve is to find a source angle profile s(t) as a function of time t so that for every angle αₗ, l=1, . . . , p, at every level v(tᵢ), i=1. . . , q, there exists a time t such that s(t)=αₗ and v(t)=v(tᵢ). In other words, the source angle is αₗ when the volume level is at v(tᵢ) for all i, l or the source covers all the angles αₗ, l=1, . . . , p exactly at all the levels v(tᵢ), i=1, . . . , q. FIG. 3 illustrates cardiac CT implemented using a constant source rotation velocity and the same set of projection angles for all the volumetric levels of interest. Further, among all the solutions s(t)'s, find one that requires the minimum time to cover all the angles for all the levels. However, the solution may not always exist in this setting. To illustrate the situation, we provide a numerical example. Let the heart volume be v(t)=r₁+r₂ sin(2πt) for some constants r₁, r₂. Suppose the volume that we are interested in is at r₁+r₂ sin [2π(5/8+m)] for integers m. Without lose of generality, let us assume that the x-ray source must scan the given heart volume at only three angles 2π(l−1)/3,l=1, 2, 3. This implies for some integers m, α(5/8+m)=2π(l−1)/3, mod 2π, l=1, 2, 3. It can be shown that there does not exist a constant x-ray source s(t)=αt so that these equations can be satisfied simultaneously.

Approximate solutions however do exist. To quantify the error, for example, we can define the minimum error between the given angles and all possible x-ray source angles at a given heart volume as e=min min max {α(5/8+m)−2π(l−1)/3, mod 2π}. The following table shows e, converted to
α>0m≧0l

degrees, and the corresponding optimal α* and m*. For instance, if the error is required to be no larger than 0.631°, the optimal α*, among all possible values (0, ∞), is 0.316π that achieves the minimum error at t=82+5/8 (unit: s). Clearly, the more accurate the solution is, the longer time the scan takes. The optimal α* is not unique. For example, α*+2kπ is also a solution, but the minimum time m+5/8 is unique.

TABLE 1
Relationship between the maximum projection
angular error and the minimum scanning time.

| | | | |
|---|---|---|---|
| α* | 0.316π | 0.036π | 0.042π |
| e | 0.631° | 0.45° | 0.045° |
| m* + ⅝ | 82 + ⅝ | 172 + ⅝ | 433 + ⅝ |

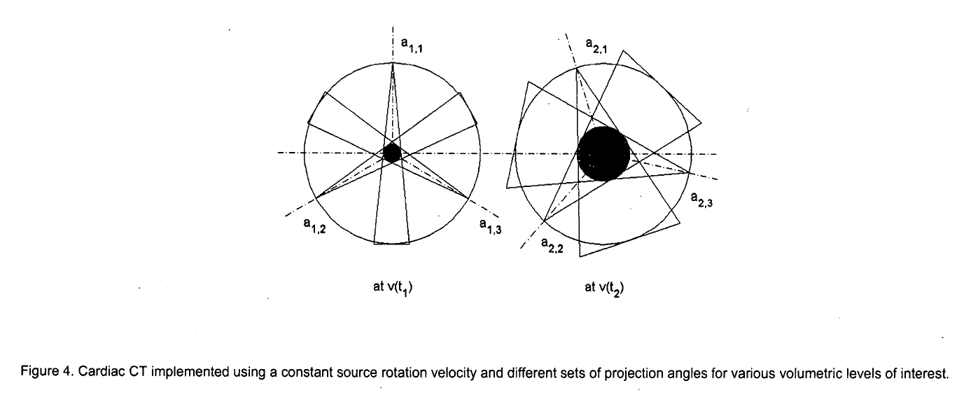

Nevertheless, we can modify the problem to allow a constant but not pre-fixed velocity solution with some angle offsets: To find a source angle profile s(t) as a fuinction of time t so that for every i at every l, there exists a corresponding time t such that s(t)=α_{i,l}=αₗ+θᵢ, and v(t)=v(tᵢ), where θᵢ is independent of the level 1. The interpretation is that at any level i, projections have to be taken at p evenly spaced angles α_{i,l}=αₗ+θᵢ, l=1, . . . , p, with the same offset angle θᵢ. However, the offset angle θᵢ at level i may not be the same as the offset angle θⱼ at level j. Further, among all the solutions s(t)'s, find one that requires the minimum time. As shown in FIG. 4, the difference lies in that it does not require that p evenly spaced angles at level i are the same as that at level j. This makes perfect sense for practical applications where an accurate image of level v(tᵢ) is reconstructed as long as p is large enough independent of whether the same angles are used for levels i and j. Surprisingly, this seeming complicated problem is solved by a constant source rotation velocity s(t) [9-11]. Let k>0 be any integer and consider

$$s ( t ) = \frac{2 π}{p Pk} t .$$

Then s(tᵢ+(l−1)Pk)=α_{i,l}=αₗ+θᵢ. This solves the problem, and further the solution with k=1 is the minimum time solution.

The solution can be also implemented by using a variable velocity X-ray source rotation [9-11]. The requirement is s(t₁+(l−1)kP)=αₗ which is an interpolation problem and k balances the minimum time needed and the maximum velocity and acceleration. One solution would be a polynomial solution s(t)=s₀+s₁t+s₂t^{2+ . . . +s}ₚ₋₁t^{p-1} so that
s₀+s₁t₁+s₂t₁²+ . . . +sₚ₋₁t₁^{p-1}=α₁
s₀+s₁(t₁+(l−2)kP)+s₂(t₁+(l−2)kP)²+ . . . +sₚ₋₁(t₁+(l−2)kP)^{p-1}=αₗ₋₁
s₀+s₁(t₁+(l−1)kP)+s₂(t₁+(l−1)kP)²+ . . . +sₚ₋₁(t₁+(l−1)kP)^{p-1}=αₗ

This polynomial solution not only matches exact p angles precisely at the level v(tₗ) but also reaches the minimum time when k=1. Recall again that a polynomial solution is just an example and any other function can also be a solution as long as the required interpolation condition is satisfied.

A.2. Quasi-Period Cardiac Motion

Recall v(t) is periodic if and only if it is completely determined by the first period, i.e., v(t)|_{tε[(l-1)P,lP)}=v(t−(l−1)P)|_{t-(l-1)Pε[0,P)}. In reality, the cardiac motion may not be periodic, especially under diseased conditions. As a result, the period for one cycle may be different from that for another cycle. However, despite the variability in the period, the cardiac volume usually alternates between the minimum and maximum volumes monotonically. Approximately, such a motion pattern can be characterized by a class of functions referred to as quasi-periodic functions. Let 0=P₀<P₁< . . . <Pₗ< . . . be a monotonically increasing sequence [9-11). The interval Pₗ₊₁−Pₗ is the lth “period” of v(t), i.e., the time to finish that specific cycle. In general, Pₗ₊₁−Pₗ≠Pₗ−Pₗ₋₁. However, we assume that v(t) is completely determined by its first period in the following sense. Let v₁(t)=v(t), tε[0, P₁), then

$$v ( t ) = {v}_{1} ( \frac{{P}_{1} - {P}_{0}}{{P}_{l} - {P}_{l - 1}} ( t - {P}_{l - 1} ) )$$

$$for$$

$$t ∈ [ {P}_{l - 1} , {P}_{l} ) .$$

Simply put, v(t) is not periodic but its appropriately scaled (expanded or compressed) version matches its profile in the first period. For such functions, the techniques that we proposed for period functions can be applied with some modifications . In particular, if all the periods are known, a control scheme can be calculated α priori that determines the order and time when the angle αₗ at the level v(tᵢ) should be imaged. The problem is that periods of a quasi-periodic heart motion are unknown α priori and have to be estimated online. Nevertheless, all the related major issues can be addressed as follows.

How to estimate periods? Suppose the period is time varying but does not change too much from one cycle to the next, it can be estimated, for example, from the ECG signal. Keep in mind, however, measurement of ECG signals is always corrupted by noises with some delay. This affects the accuracy of the estimation but can be addressed properly. For example, this can be done as follows. Suppose for the (1−1)th period, Pₗ₋₁−Pₗ₋₂, is estimated, and we need to estimate the lth period Pₗ−Pₗ₋₁ or equivalently to estimate

$${α}_{l} = \frac{{P}_{1} - {P}_{0}}{{P}_{l} - {P}_{l - 1}} .$$

Now, a new observation

$$v ( \overline{t} ) = {v}_{1} ( \frac{1}{{α}_{l}} ( \overline{t} - {P}_{l - 1} ) )$$

is obtained, this gives rise to the information on α₁=( t̅−Pₗ₋₁)/v⁻¹(v( t̅)), where v⁻¹ denotes the inverse function of v. To reduce the effect of noises, an average of several such observations could be used. If necessary, other estimation and prediction algorithms that were developed by our and other teams [13-19] can be used for controlled cardiac CT, including algorithms that are based on the Hammerstein model, non-parametric model, variable gain model and approximate linear model, respectively.

How to design the control scheme based on the estimates of the periods? There is always a chance that the estimation error is larger than a pre-specified threshold. For example, if in the lth period the error is larger than the threshold, then the images taken during the lth period may become non-usable. We can address this problem using a monitoring mechanism in the system that compares the predicted and the actual periods (see FIG. 1). If a larger error occurs, the missing data has to be re-sampled later, using a similar control scheme to fill in the missing gaps in the desirable projection region.

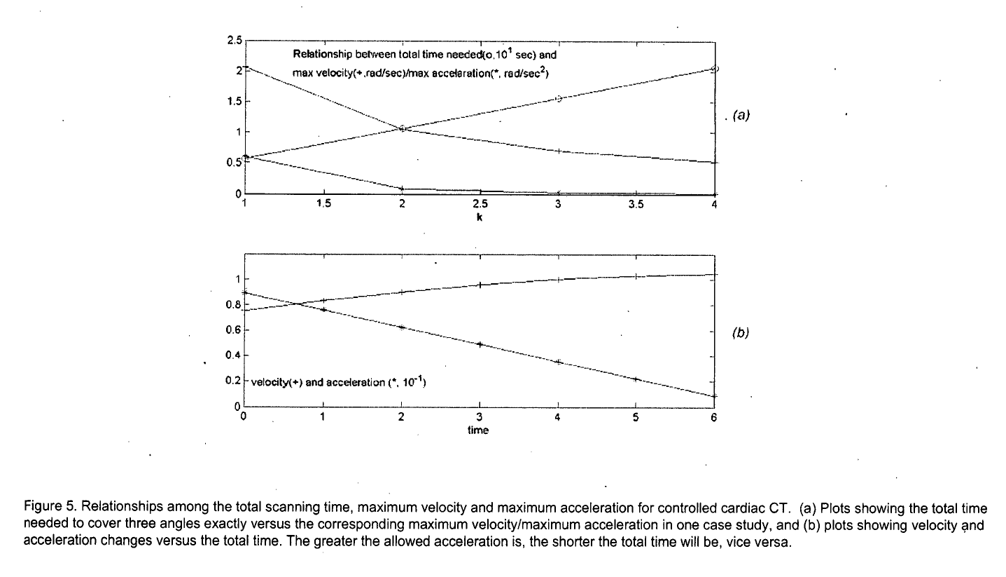

In the solution given above, the variable k is used to balance the minimum time required and maximum velocity and acceleration. As k increases, the required scan time increases and at the same time the maximum velocity/acceleration of s(t) decreases. Addition of k in the solution is important practically. There are other ways to take velocity/acceleration into consideration. For instance, instead of usual polynomial interpolation, Fejer or other similar types of interpolations [12,13] that have constraints on its derivatives can be used. Since velocity and acceleration are the first and second order derivatives respectively, Fejer interpolation effectively sets constraints on the allowable maximum velocity and acceleration. It is important to emphasize again that hardware constraints must be taken into account in practice, which can be achieved by balancing between the scan time and the maximum speed and maximum acceleration using our invented control scheme, as demonstrated in FIG. 5.

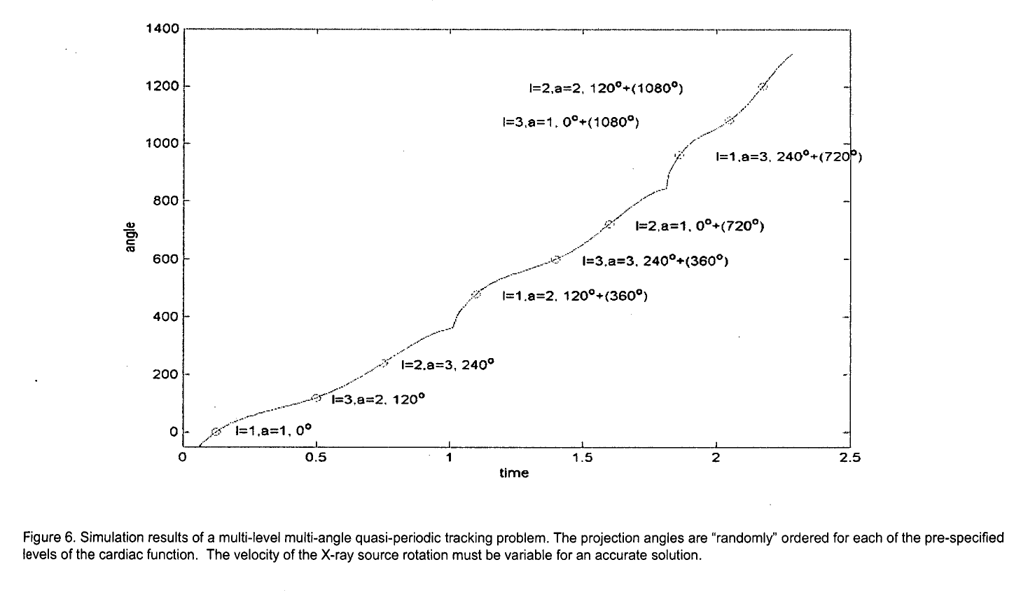

To illustrate the method, we give an example. Consider a heart volume v(t)=1−0.5 Cos(2*π*t/H) for three unknown periods H=1, 0.8 and 0.5. This is no longer periodic but quasi-periodic. Suppose that three heart levels v(t)=0.64, 1, 1.5 and three angles at 0, 120 and 240 degrees are specified. By using the method discussed above with a polynomial interpolation, FIG. 6 shows the computer simulation results based on the estimates of the unknown periods. Clearly, all angles at all three heart levels are imaged. The goal is achieved using a variable velocity X-ray source. A constant velocity X-ray source either fails to do that or takes too long to even have a good approximate solution, as discussed in Table 1.

As another embodiment, CCCT can be implemented using a monitoring mechanism and a control mechanism which is manual, semi-automatic, automatic, or in a mixed mode. This combined setup monitors the data completeness status, identifies missing projections, and adjusts the source rotation velocity/acceleration of the scanner to make up these missing projections in an optimal or heuristic way. For example, for each cardiac state/phase, covered and not covered projection angles can be graphically displayed in a disk like panel, in which the current heart motion frequency, the source angular position, velocity and acceleration can be also graphically shown. Based on this type of visual input, an operator can steer the source using, for example, arrow keys, subject to some physical constraints. A sample heuristic rule for steering the source is that the source be driven to cover the missing projection that takes the shortest source motion time. More sophisticated rules can be constructed in a similar spirit. This type of rules can be automated as preferred; for example, using an interpolation based control strategy similar to what discussed above.

A.3. Approximate Matching to Desirable Projection Angular Positions

Another very useful problem is the non-exact matching case. In the above analysis, the p angles have to be exactly covered at q volume levels. In reality, the exact angles and precise levels are not necessary as long as the errors are small. By non-exact matching, we mean at each level images do not have to reflect the volume level v(αₗtᵢ+Pₗ₋₁) exactly as long as it stays in a small neighborhood of the targeted level. Likewise, the projection angles do not have to be exactly at αₗ but it suffices for them to be in a small neighborhood of αₗ.

To this end, at each level i, let the error bound δ>0, the corresponding times t_{-i,l}≦tᵢ≦ t̅_{i,l} can be found

$${max}_{t ∈ [ {α}_{l} {\underline{t}}_{i , l} + {P}_{l - 1 ,} {α}_{l} \overline{t } i , l + {P}_{l - 1} ]} \vert v ( t ) - v ( {α}_{l} {t}_{i} + {P}_{l - 1} ) \vert \leq δ$$

Furthermore, let us define τ_{i,l}=[αₗt_{i,l}+Pₗ₋₁,αₗ t̅_{i,l}+Pₗ₋₁] and

$${Δ}_{i , l} [ {min v ( t )}_{t ∈ [ {α}_{l} {\underline{t}}_{i , l} + {P}_{l - 1 ,} {α}_{l} {\overline{t}}_{i , l} + {P}_{l - 1} ]} , max v ( t ) ]$$

The neighborhood of v(αₗ tᵢ+Pₗ₋₁) is Δ_{i,l} with the corresponding permissible time interval τ_{i,l}. That is, v(αₗ tᵢ+Pₗ₋₁)εΔ_{i,l}, max_{tετ}_{_{i,l}}|v(t)−v(αₗtᵢ+Pₗ₋₁)|≦δ and τ_{i,l} is the time interval in which the images for any angle αₗ, l=1, . . . , p can be taken at the level i. By the same token, in a non-exact case, the volume level i can be defined as Lᵢ={Δ_{i,l}, Δ_{i,2}, . . . , Δ_{i,p} . . . } or in time domain Tᵢ={τ_{i,l}, τ_{i,2}, . . . , τ_{i,p}, . . . }. Now, the non-exact match problem can be defined as to find an x-ray source angular position s(t) that satisfies |s(t)−αₗ≦ε for tετ_{i,k}, where ε>0 is the given angle error bound. The resultant s(t) significantly reduces the total data acquisition time needed to cover all the projection angles at each of the desired cardiac volume levels. The control algorithms can be accordingly developed, for example, using the numerical search technique.

B. Image Reconstruction

B.1. Feldkamp-Type Reconstruction

As far as the imaging part is concerned, we may, for example, use the Feldkamp cone-beam image reconstruction algorithm or the generalized Feldkamp algorithm developed by Wang et al. [20,21]. This algorithm can be written as:

$$g ( x , y , z ) = \frac{1}{2} {\int }_{0}^{2 π} \frac{{D}^{2} ( β )}{{( D ( β ) - s )}^{2}} {\int }_{- ∞}^{∞} R ( p , ζ , β ) h ( \frac{D ( β ) t}{D ( β ) - s} - p ) \frac{D ( β )}{\sqrt{{D}^{2} ( β ) + {p}^{2} + {ζ}^{2}}} ⅆ p ⅆ β ,$$

where g(x,y,z) is a reconstructed 3D image, D(β) the distance between the source and the z-axis of the reconstruction system in which an object of interest η(x,y,z) is located, β the source rotation angle relative to the z axis, R(p,ζ, β) cone-beam projection data of η(x,y,z),

$$\{ \begin{matrix}ζ = \frac{D ( β ) z}{D ( β ) - s} \\ p = \frac{D ( β ) t}{D ( β ) - s} ,\end{matrix} \\ \{ \begin{matrix}t = x cos β - y sin β \\ s = - x sin β - y cos β .\end{matrix}$$

Let us assume that D(β) is a constant, the approximate reconstruction procedure consists of the following three steps:
1. Weight cone-beam projection data

$${R}^{′} ( p , ζ , β ) = \frac{D}{\sqrt{{D}^{2} + {p}^{2} + {ζ}^{2}}} R ( p , ζ , β ) ;$$
2. Filter the weighted data Q(p, ζ, β)=R′(p, ζ, β)*h(p);
3. Back-project the filtered data

$$g ( x , y , z ) = {\int }_{0}^{2 π} \frac{{D}^{2}}{2 {( D - s )}^{2}} Q ( p , ζ , β ) ⅆ β .$$

Note that the algorithm does allow non-uniform sampling patterns along a scanning circle. If the cone-angle becomes larger, more sophisticated algorithms may be adapted, including both exact and approximate algorithms with circular or non-circular scanning trajectories [22-28].

B.2. Grangeat-Type Reconstruction

Collected CCCT data can be fed into any representative CT algorithm after appropriate data interpolation, which should be clear to those who practice in the cardiac CT field. As another example of the method for cone-beam CCCT, we can use the Grangeat-type reconstruction [29-31]. While traditional half-scan cone-beam algorithms are in the Feldkamp framework, Grangeat-type half-scan cone-beam algorithms were subsequently developed in the circular and helical scanning cases. The Grangeat-type framework promises better quality reconstruction from an incomplete dataset after missing data are appropriately estimated. We can utilize the explicit Radon space information available in collected cone-beam data, perform appropriate data filling in the shadow zone of the Radon space, and suppress various artifacts in the final reconstruction. In the circular half-scan case, the original Grangeat formula can be modified into the following half-scan

$$version : \frac{\partial }{\partial ρ} Rf ( ρ {h}^{ρ} ) = {\sum }_{i = 1}^{2} {ω}_{i} ( ρ {h}^{ρ} ) \frac{1}{{cos}^{2} β} \frac{\partial }{\partial s} {\int }_{- ∞}^{∞} \frac{SO}{SA} Xf ( s ( ρ {h}^{ρ} ) ) ⅆ t , where \\ {ψ}_{1} ( ρ , θ , φ ) = φ + {sin}^{- 1} [ \frac{ρ}{SO sin θ} ] and {ψ}_{2} ( ρ , θ , φ ) = φ + π - {sin}^{- 1} [ \frac{ρ}{SO sin θ} ]$$

are two meridian plane angle functions, depending on a characteristic point in the Radon space. The scanning angle ψ varies from 0 to π+2γₘ, where γₘ is the cone angle. In the circular full-scan case, for any characteristic point not in the shadow zone or not on its surface there exist a pair of detector planes specified by the above two angle functions. However, in our half-scan case, such the dual planes are not always possible. When the dual planes are found, we are in a doubly sampled zone. When one of them is missing due to the half-scan, we are in a singly sampled zone. Relative to the full-scan, the shadow zone is increased due to the reduction in the amount of cone-beam data.

In the context of this invention, we can adapt our half-scan Grangeat-type reconstruction for cardiac CT from data collected from x-ray source positions pseudo-randomly distributed along a scanning circle. This adaptation can be done in a number of ways. The main challenge is how to design an best interpolation strategy so that the image quality, especially the temporal resolution, can be optimized. For that purpose, we can model time-varying Radon information as well to interpolate for missing data in both the spatial and temporal domains. We can use various linear interpolation methods to estimate missing derivative data in the shadow zone corresponding to different cardiac phases. The heuristics behind our choice of the linear interpolation approach is that the derivative Radon data of many geometrically regular objects, such as ellipsoids or tetrahedrons, are piece-wise linear along the ρ direction. Therefore, the linear interpolation method may help effectively recover missing data. Then, we can equip this interpolation method with some adaptive capabilities. That is, the slope of the linear interpolation may vary region by region, according to prior knowledge such as the boundaries of the cardiac components derived from earlier reconstructions.

B. 3. Examples on Controlled Cardiac CT

The essential feature of this invention is to synchronize the x-ray source rotation and data acquisition optimally so that the dynamics of a beating heart can be captured with minimum distortion. This type of synchronization can be implemented in a number of ways. In a simple case, we can use an arbitrary but pre-selected source rotation speed. In a complicated case, we have to steer the source rotation with a variable acceleration.

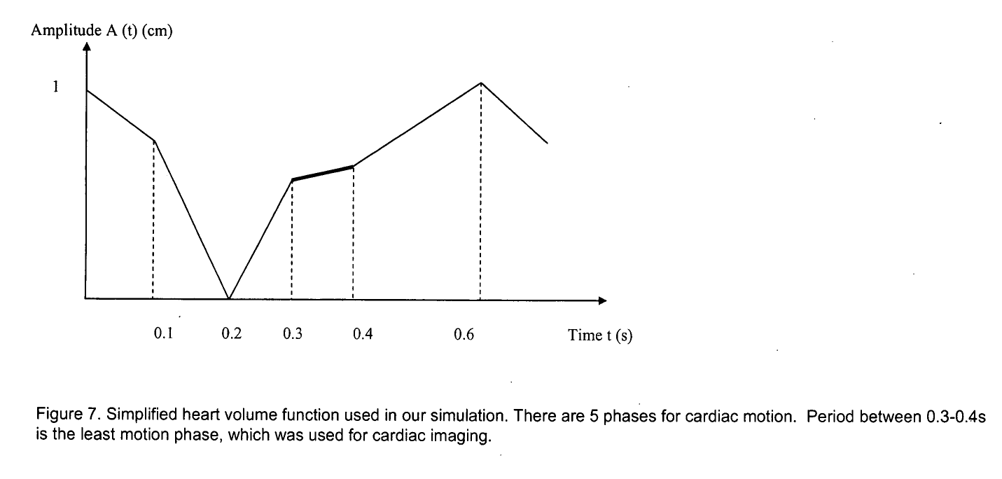

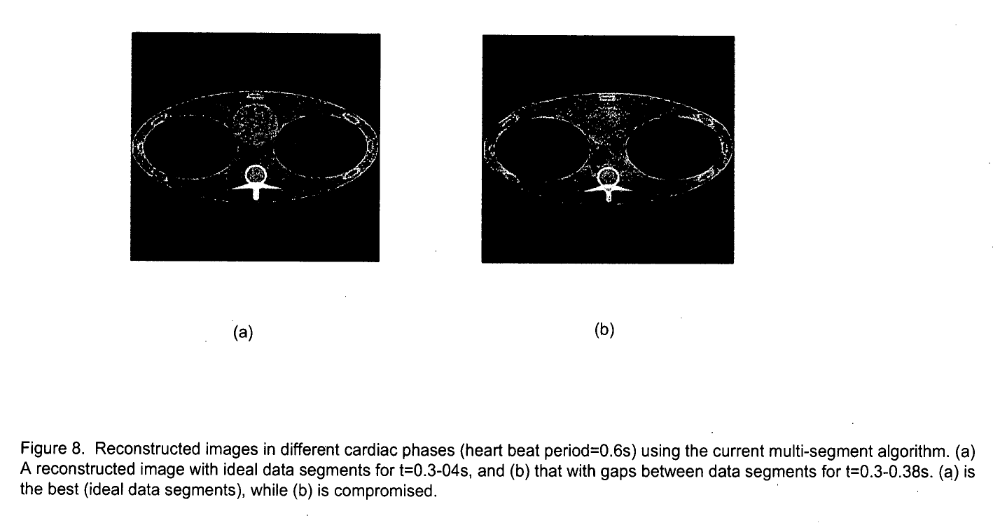

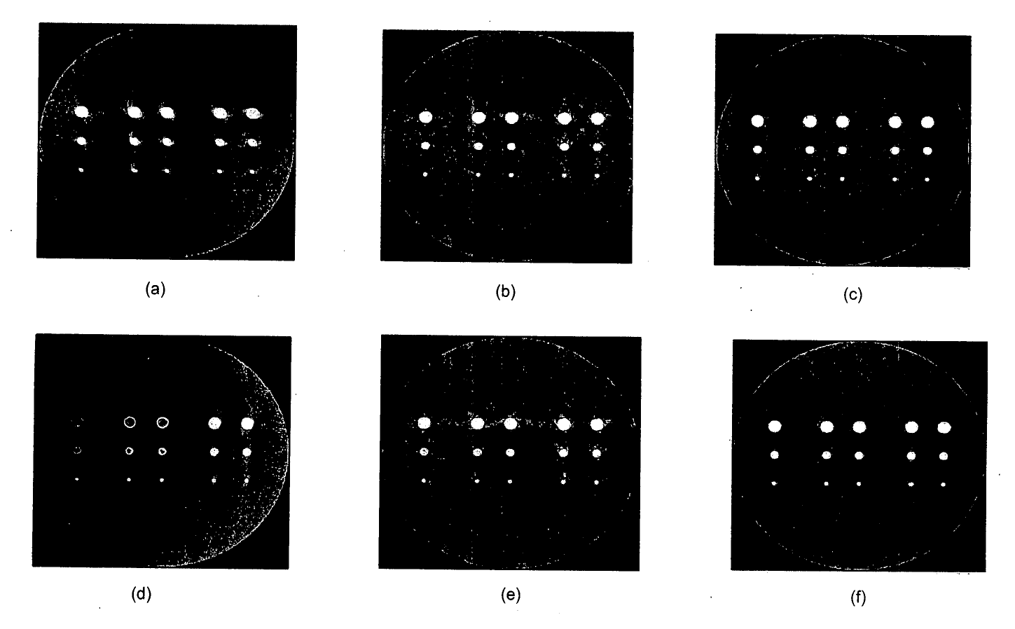

Without loss of generality, here we compare the results obtained with the existing schemes of fixed source rotation with our method for a selectable source rotation speed [11]. Several numerical tests were carried out using two cardiac phantoms. They are the thorax phantom and the motion phantom. The thorax phantom consists of parts simulating lungs, heart, aorta, ribs, spine, stemum and shoulders, respectively. There are 271 basic objects in the thorax phantom, constructed by spheres, cylinders and boxes. A heart is represented by an ellipsoid. The aorta is composed of three cylinders. For simplicity, the motion of the left ventricle is divided into 5 phases, depicting the basic systolic and diastolic processes, as shown in FIG. 7. The period of the cardiac motion was set to 0.6 s with the motion amplitude being 1 cm along each semi-axis of the heart. The motion phantom contained three rows of cylindrical patterns with the diameters being 1, 2 and 3 mm, respectively. The second and fourth columns were vibrated along the horizontal direction according to a sinusoid function. The motion amplitude was set to 5 mm. Other relevant parameters include the heart period P=0.6 s, the source rotation period Pc=0.5 s, temporal resolution τ=0.1 s, allowing an ideal combination of data segments. The circular fan-beam scanning was assumed with 400 detectors and 360 views. A 256 by 256 image matrix was reconstructed. For the thorax phantom, the reconstructed images are shown in FIG. 8 corresponding to the cases of multi-segment combinations without any gap ((a), τ=0.1 s), with gaps ((b), τ<0.1) and with overlaps ((c), τ>0.1 s), respectively. FIG. 8(a) has the best quality because the data segment was ideal. FIG. 8(b) is unsatisfactory and FIG. 8(c) is a little distorted due to the gaps and overlaps. This simulation shows that better image can be only achieved in ideal segment. However, temporal resolution is fixed and can not be improved in current cardiac CT. In the following, three types of simulations are provided.

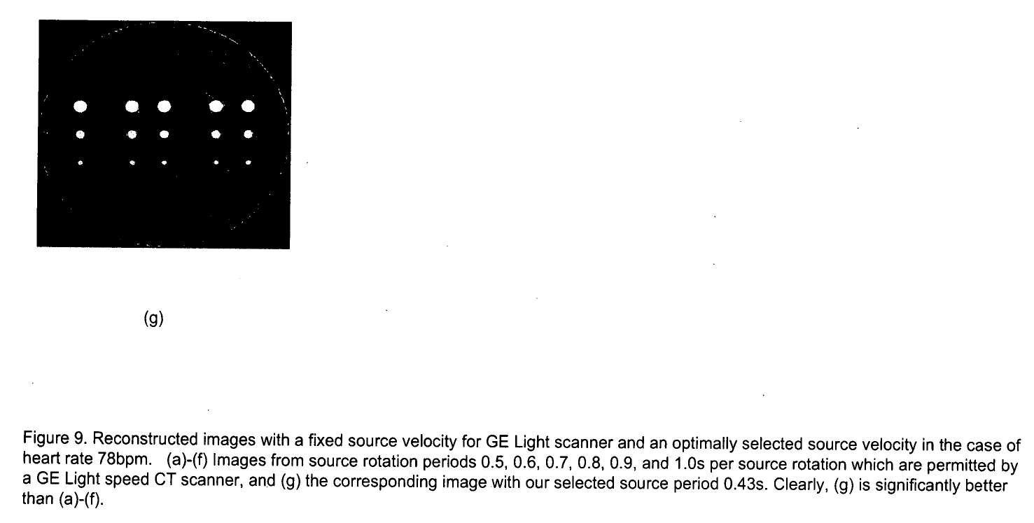

In the first simulation, we consider the GE Light Speed scanner because it can offer more choices of velocities than others. Six fixed velocities were assumed, including 0.5, 0.6, 0.7, 0.8, 0.9, and 1.0 s per rotation as provided by GE Light Speed scanner. We reconstructed images based on these velocities and compared these images with ones reconstructed using the selectable velocity method. When the heart rate was 78 bpm (beats per minute) or the heart period was 0.77 s. None of the available source rotation periods 0.5 s, 0.6 s, 0.7 s, 0.8 s, 0.9 s and 1.0 s would provide an ideal multi-segment combination. Hence, needed multi-segments must be assembled from available data after necessary interpolation. FIG. 9 shows the reconstructed image over 5 heart cycles using the current and our methods. Clearly, the image reconstructed by the selected rotation period 0.43 s (FIG. 9(g)) is better than the images reconstructed with fixed periods 0.5, 0.6, 0.7, 0.8, 0.9, and 1.0 s (FIG. 9(a)-(f)) because the data segment was ideal with the selected source rotation velocity but it was not ideal with any fixed velocity.

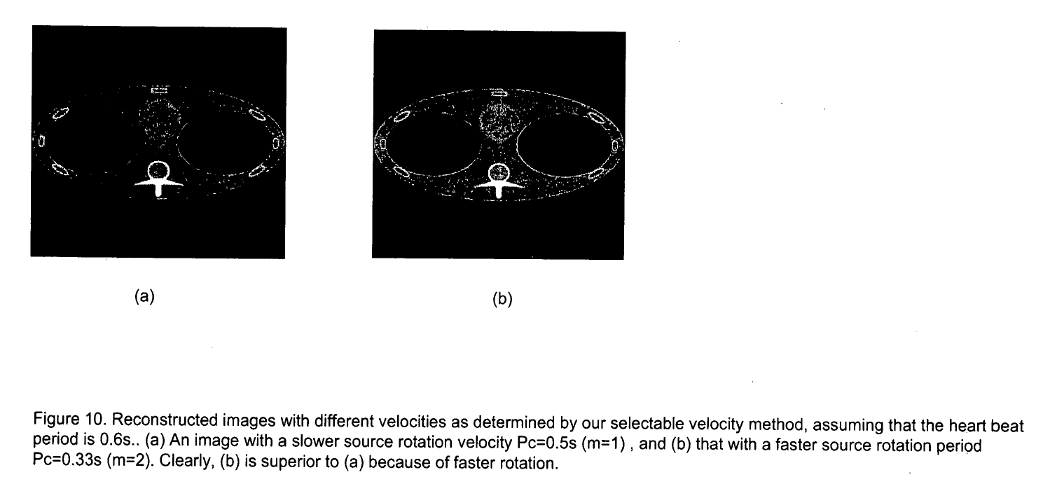

In the second simulation, different velocities were selected by selecting different adjustable parameters in the formula. Generally speaking, the faster the rotation is, the better the temporal resolution is. For example, the setting of P=0.6 s, and m=1 implies Pc=0.5 s. If m=2, then Pc=0.33 s. Some representative images are illustrated in FIG. 10. FIG. 10(b) is better than FIG. 10(a) because the velocity was higher for FIG. 10(b). On the other hand, commercially available CT scanners do not allow a free choice of the source rotation velocity to optimize the image quality. The simulation shows that faster rotation would result in better images using our method.

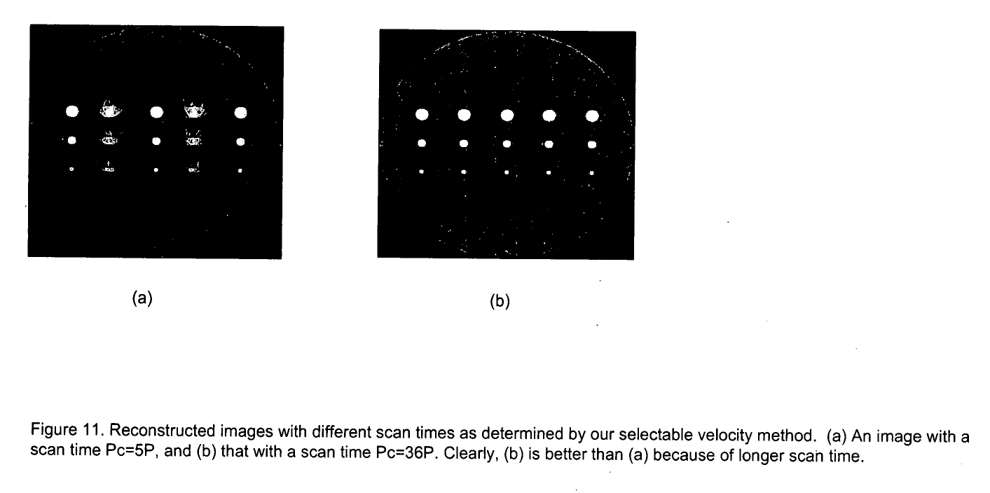

In the third simulation, the images were reconstructed from scans of different temporal spans. For a fixed source rotation velocity, even if ideal data segments were collected, temporal resolution would not be improved further by prolonging the scanning time. On the other hand, a higher temporal resolution would be achieved using our selectable velocity method if a longer scanning time was taken. Some simulation results are given in FIG. 11. FIG. 11(b) is much better than FIG. 11(a) because the total time was longer for FIG. 11(b). This example shows that the image quality can always be improved by extending the total scanning time for our selectable velocity method. On the other hand, the image quality could not be improved by prolonging scanning time for a fixed velocity CT.

As mentioned above and demonstrated in our simulation, the our variable velocity method also outperformed the fixed velocity method in terms of image quality in both the periodic and quasi-periodic cases. The major merits with the variable velocity method include a significantly reduced scanning time and a unique capability of dealing with a quasi-periodic cardiac motion pattern. Once a better dataset is collected using any of our proposed techniques, the image reconstruction process is not so much different, since it is nothing but image reconstruction from projections at pseudo-randomly distributed angular positions. Therefore, we will not present more simulation results here, for sake of brevity.

1. The controlled cardiac computed tomography (CCCT) methodology and techniques that consist of all or some of the following components:
(i) a method for monitoring and predicting the cardiac motion pattern;
(ii) a method for identifying missing projections needed for a desirable cardiac reconstruction quality;
(iii) a mechanism for steering the x-ray source rotation in reference to the cardiac motion pattern, the data incompleteness and the current source parameters (position, velocity, etc.) for collection of needed projection data;
(iv) a data acquisition system controlled by the said mechanism;
(v) an image reconstruction algorithm that reconstructs cardiac images from the data collected by the said data acquisition system;

2. The methods and techniques described by claim 1 in which the control mechanism uses a constant source rotation velocity but different sets of projection angles for various cardiac levels/states;

3. The methods and techniques described by claim 1 in which the control mechanism uses a variable source rotation velocity for collection of needed projection data;

4. The methods and techniques described by claim 1 in which the control mechanism uses an interpolation based scheme, and is featured by a variable source rotation velocity for collection of needed projection data;

5. The methods and techniques described by claim 1 in which the control mechanism has a variable that balances the scanning time and maximum velocity/acceleration.

6. The methods and techniques described by claim 1 in which the control law is derived according to various motion models: periodic, quasi-periodic or non-periodic;

7. The methods and techniques described by claim 1 in which the control implementation is based on robust control, adaptive control, optimal control, nonlinear control and/or other types of control methods and techniques;

8. The methods and techniques described by claim 1 in which the data acquisition process uses a circular, helical, saddle curves or other scanning trajectories;

9. The methods and techniques described by claim 1 in which the reconstruction algorithm is analytic and/or iterative (such as filtered backprojection, ART, EM, OSEM);

10. The system that utilizes the methods and techniques described by claim 1;

11. The system defined by claim 10 that utilizes a multi-source/detector design;

12. The system defined by claim 10 that is for CT imaging of a patient;

13. The system defined by claim 10 that is for CT imaging of an animal;

14. The system defined by claim 10 that is for micro-CT imaging of a small animal;

15. The system defined by claim 10 that is based on the rotation of an animal instead of the rotation of the x-ray source(s);

16. The method defined by claim 1 that integrates control and imaging algorithms based on ECG signals or likes in cardiac CT.

17. The method defined by claim 1 that is implemented using a monitoring mechanism and a control mechanism which is manual, semi-automatic, automatic, or in a mixed mode. This combined setup monitors the data completeness status, identifies missing projections, and adjusts the source rotation velocity/acceleration of the scanner to make up these missing projections in an optimal or heuristic way.

18. The system defined by claim 10 that is implemented using a monitoring mechanism and a control mechanism which is manual, semi-automatic, automatic, or in a mixed mode. This combined setup monitors the data completeness status, identifies missing projections, and adjusts the source rotation velocity/acceleration of the scanner to make up these missing projections in an optimal or heuristic way.

19. The method defined by claim 1 except that the beating heart is replaced by another periodic or quasi-periodic moving structure, relevant to another biomedical, industrial application or applications in other areas.

20. The system defined by claim 10 except that the beating heart is replaced by another periodic or quasi-periodic moving structure, relevant to another biomedical, industrial application or applications in other areas.
